(19) United States
(12) Patent Application Publication
(10) Pub. No.: US 2001/0053795 A1
(43) Pub. Date: Dec. 20, 2001

(54) Paradoxical pharmacology

(76) Inventor: Richard A. Bond, Houston, TX (US)

Correspondence Address: FULBRIGHT & JAWORSKI L.L.P., Melissa W. Acosta, 1301 McKinney, Suite 5100, Houston, TX, 77010-3095, US

(21) Appl. No.: 09/885,183

(22) Filed: Jun. 20, 2001

Related U.S. Application Data
(60) Provisional application No. 60/212,579, filed on Jun. 20, 2000.

(51) Int. Cl. A61K 31/223

(52) U.S. Cl. 514/551

(57) ABSTRACT

The present invention relates to a method of treatment of a disease comprising the step of administering to an animal a paradoxical pharmacologic agent, wherein the agent restores compensatory mechanisms or activates redundant mechanisms. More particularly, the invention relates to methods of treatment for hypertension.

This application claims priority to U.S. Provisional Application No. 60/212,579, which was filed on Jun. 20, 2000.

### FIELD OF INVENTION

This invention relates to the use of contraindicated drugs to treat and/or prevent disease. More particularly, it relates to the prophylactic and therapeutic treatment of a disease or condition.

### BACKGROUND OF INVENTION

Medicine has a monolithic view for the treatment of disease, i.e., assisting or helping to correct a malfunctioning system. By accident, medicine has increased stress upon the system resulting in treatment. For example, the class of drugs termed, ‘β-blockers’, were originally contraindicated in patients with congestive heart failure (CHF). These drugs, officially termed, β-adrenoceptor antagonists, block subtypes (the β-subtypes) of the receptors for adrenaline and noradrenaline. In the heart, β-adrenoceptors increase contractility and rate when stimulated by adrenaline or noradrenaline. In CHF the heart begins to fail as a pump and has difficulty meeting the blood supply demands of the organs. The body's response to the diminished output from the heart is to increase the release of adrenaline and noradrenaline, thereby stimulating cardiac contractility. Thus, the thought that to further impede contractility by blocking β-adrenoceptors would only exacerbate the disease was not logical. However, in 1975, a Swedish study administered β-blockers to patients with CHF. It was later discovered that the body's response of increasing the release of adrenaline and noradrenaline, while good short-term, also led to desensitisation of the cardiac β-adrenoceptors over the long-term (Bristow, 1982). Now, there is widespread acknowledgment of β-blockers as an essential component of heart failure treatment. However, the question remains it is the protection of the β-adrenoceptors that increased contractility, or is it shutting down a struggling system and forcing or restoring other compensatory mechanisms.

There are reports that Alzheimer's disease (AD) and other neurodegenerative dementias can be associated with a hypofunction of the dopamine system (Zubenko, 1999; Walker, 1999; Joyce, 1998). There are also reports of a decreased incidence of Alzheimer's and other neurodegenerative disorders in schizophrenic patients (Arnold, 1994; Arnold, 1998). The mainstay of pharmacologic treatment for schizophrenia is dopamine antagonists. In 1994, Arnold et al., as well as those of Purohit et al., prompted the speculation that the neurobiology of schizophrenia (or even its treatment with antipsychotic medication) may ‘protect’ against the development of AD pathology. Since schizophrenia is a complex disease(s), and often misdiagnosed, it would appear that the simpler explanation is the treatment is producing the decreased incidence.

Another isolated instance where paradoxical pharmacology has become accepted is the use of stimulants such as methylphenidate to treat hyperactivity in children. Psychiatry/psychology appears to also hold clues that paradoxical approaches can produce beneficial results in human response. Thus, branches of this discipline have successfully employed reverse psychology and paradoxical interventions as treatment modalities.

A natural paradoxical response occurs during exercise. During strenuous aerobic exercise, there is an increase in the levels of endogenous catecholamines (epinephrine and norepinephrine) resulting in an increase in systolic pressure. Yet, in some hypertensive patients, regular exercise programs are encouraged to lower blood pressure. Thus, chronic, intermittent increases in blood pressure, as in exercise, produce a net decrease in blood pressure over the long term.

One possible general hypothesis for why paradoxical pharmacology may work is the difference of the chronic versus the acute effect of drugs. In the case of ‘β-blockers’ in heart failure, initially the patient often reports feeling worse, but then there is improvement of function and a decrease in mortality. Clearly, the chronic response differs from the acute response. There are numerous other examples of differences between acute and chronic response. For example, classical antidepressants often take weeks of treatment to produce their effects. Furthermore, acute and chronic effects of drugs often produce opposite effects. This is particularly true for receptor mediated events. For example, acute agonist exposure may produce activation of receptors and increased signaling, while chronic exposure may produce desensitization and decreased signaling. Finally, to again draw the parallel with exercise, acutely exercise raises blood pressure, while chronically there is a decrease in blood pressure.

Thus, chronic and acute responses often differ substantially. However, most often contraindications for drugs are predicted based on the assumption that the chronic effect will be simply a more prolonged version of the acute effect. Perhaps the lesson with ‘β-blockers’ in heart failure can teach something. When trials with ‘β-blockers in heart failure were first started, the doses were too high and the drugs were poorly tolerated. It was discovered that it was necessary to start at a low dose and gradually increase the dose over a period of weeks.

Furthermore, the present invention is the first to use the concept of increased stress upon the system to treat and possible prevent diseases or conditions. It is noteworthy that the present invention is the first systematic approach to study paradoxical pharmacology. Until the present disclosure, any contraindicated evidence was obtained as a consequence of the medical field trying to “help and assist the ailing system”, suggesting that this invention is indeed non-obvious.

Thus, if acute versus chronic responses are often opposite in nature, and if the contraindications are based on the acute effects, then there is a suggested list of where basic research can begin to look for clues to investigate paradoxical pharmacology. It is the list under ‘Contraindications’, because the opposite of contraindicated is indicated. This is the list where one found β-blockers in congestive heart failure just a short time ago.

### SUMMARY OF THE INVENTION

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

A specific embodiment of the present invention is a method of treatment of a disease comprising the step of administering to an animal a paradoxical pharmacologic agent, wherein the agent restores compensatory mechanisms or activates redundant mechanisms. Specifically, the disease is selected from the group consisting of hypertension and diabetes.

In specific embodiments, the paradoxical pharmacologic agent is selected from the group consisting of an agonist, an antagonist or an inverse agonist.

In further embodiments, the paradoxical pharmacologic agent is administered to a mucosal surface of the animal. In yet another embodiment, the paradoxical pharmacologic agent is administered parenterally to the animal.

A specific embodiment of the present invention is that the paradoxical pharmacologic agent is a prophylactic treatment. Further, the prophylactic treatment requires chronic administration of the paradoxical pharmacologic agent.

In specific embodiments, the paradoxical pharmacologic agent is given in a single dose or a series of doses. Specifically, the doses may be given daily, weekly, or monthly.

Another specific embodiment is a method of treatment of hypertension comprising the step of administering to an animal a paradoxical pharmacologic agent, wherein the agent restores compensatory mechanisms or activates redundant mechanisms. Specifically, the paradoxical pharmacologic agent is an agonist or an inverse agonist. More particularly, the agonist is a pressor agent. In specific embodiments, the pressor agent is selected from the group consisting of phenylephrine, angiotensin II, epinephrine and norepinephrine.

In yet another embodiment, the pressor agent is a prophylactic treatment of hypertension. More particularly, the prophylactic treatment requires chronic administration of said paradoxical pharmacologic agent.

In a further embodiment, the method further comprises the administration of a combination of at least one paradoxical pharmacologic agent and at least one non-paradoxical pharmacologic agent.

In yet another specific embodiment, the method further comprises the administration of a combination of at least one paradoxical pharmacologic agent and at least one non-paradoxical pharmacologic agent.

### DESCRIPTION OF THE DRAWING

The following drawings form part of the present specification and are included to further demonstrate certain aspects of the present invention. The invention may be better understood by reference to one or more of these drawings in combination with the detailed description of specific embodiments presented herein.

FIG. 1 illustrates the two-state model of receptor theory where there is an equilibrium between an inactive state of the receptor (R) and the active state of the receptor (R*).

FIG. 2 illustrates the mean blood pressure of spontaneously hypertensive rats treated with vehicle (SHR veh) and with a combination of epinephrine and norepinephrine (SHR NE/E). The white bars represent mean blood pressure on the last day of infusion. The black bars represent the mean blood pressure 2 days after infusion. The stippled bars represent the mean blood pressure 3 days after infusion.

FIG. 3 illustrates the mean blood pressure in spontaneously hypertensive rats after L-NAME infusion. SHR NE/E represents rats treated with norepinephrine and epinephrine for 28 days and SHR VEH represents rats treated with vehicle for 28 days.

### DETAILED DESCRIPTION OF THE INVENTION

It is readily apparent to one skilled in the art that various embodiments and modifications may be made to the invention disclosed in this application without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

In the two-state model of receptor theory, the term “agonist” as used herein is defined as a substance that has an affinity for the active state of a receptor and thereby preferentially stabilizes the active state of the receptor or a compound, including, but not limited to, drugs, hormones and neurotransmitters, which produces activation of receptors. Irrespective of the mechanism(s) of action, an agonist produces activation of receptors.

In the two-state model of receptor theory, the term “antagonist” as used herein is defined as a substance that does not preferentially stabilized either form of the receptor, active or inactive, or a compound, including, but not limited to, drugs, hormones, and neurotransmitters, which prevents or hinders the effects of agonists and/or inverse agonists. Irrespective of the mechanism(s) of action, an antagonist prevents or hinders the effects of agonists and/or agonists.

In the two-state model of receptor theory, the term “inverse agonist” as used herein is defined as a substance that has an affinity for the inactive state of a receptor and thereby preferentially stabilizes the inactive state of the receptor, or a compound, including, but not limited to, drugs, hormones, and neurotransmitters, which produces inactivation of receptors and/or prevents or hinders activation by agonists. Irrespective of the mechanism(s) of action, an inverse agonist produces inactivation of receptors and/or prevents or hinders activation by agonists.

The term “contraindicated” as used herein is defined term to indicate the inadvisability of a medical drug to treat a particular disease. Contraindicated may be absolute, “do not use”, or relative, “use with caution.”

The term “disease” as used herein is defined as a disorder or condition that disrupts body functions, systems or organs. Exemplary diseases include, but are not limited to, asthma, hypertension, pain, diabetes, Alzheimer's, and Parkinson's.

The term “paradoxical pharmacologic agent” as used herein is defined as a drug or agent that is considered to be contraindicated to treat and/or prevent a disease or condition.

The term “prophylactic” as used herein is defined as a drug or agent which acts to prevent a disease or condition, e.g., a vaccine.

The term “pressor agent” as used herein is defined as a drug that increases blood pressure.

The term “treatment” as used herein is defined as the use of a drug as a therapeutic or prophylactic therapy. A skilled artisan is cognizant that “treatment” refers to the management and care of an animal for the purpose of combating a disease or condition. Thus, the term treatment as used herein is all inclusive of the acts of curing a disease, preventing a disease or merely managing a disease.

The term “therapeutic” as used herein is defined as a drug or agent, which acts to treat a disease or condition.

The term “receptor” as used herein is defined as a cellular structure, usually, but not limited to, protein to which compounds, including, but not limited to, drugs, hormones, and neurotransmitters, bind to produce or prevent production of a cellular response.

A specific embodiment of the present invention is a method of treatment of a disease comprising the step of administering to an animal a paradoxical pharmacologic agent, wherein said agent restores compensatory mechanisms or activates redundant mechanisms. Exemplary diseases include, but are not limited to, hypertension and diabetes.

One skilled in the art realizes that fail-safe or redundant mechanisms govern crucial functions in an animal. In addition to each organ system having the capacity to function at levels beyond the usual day-to-day demands, there are duplicative or overlapping controls to provide excess regulatory capacity in critical situations. For example, two hormones secreted from two different tissues, sometimes in response to different conditions, produce the same end result, e.g., conversion of liver glycogen to blood glucose can be signaled by at least two hormones (glucagon and epinephrine). Redundant mechanisms assure that critical processes occur and also offer the opportunity for flexibility and subtle fine tuning of processes. One of the important implications of redundancy is that in a disease state, partial or complete failure of one of the mechanisms can be compensated by increased reliance on another mechanism. Thus, a skilled artisan recognizes the importance of redundant mechanisms and is capable of selecting potential paradoxical pharmacologic agents that could be used to activate or restore a compensatory mechanism or activate or restore a redundant mechanism.

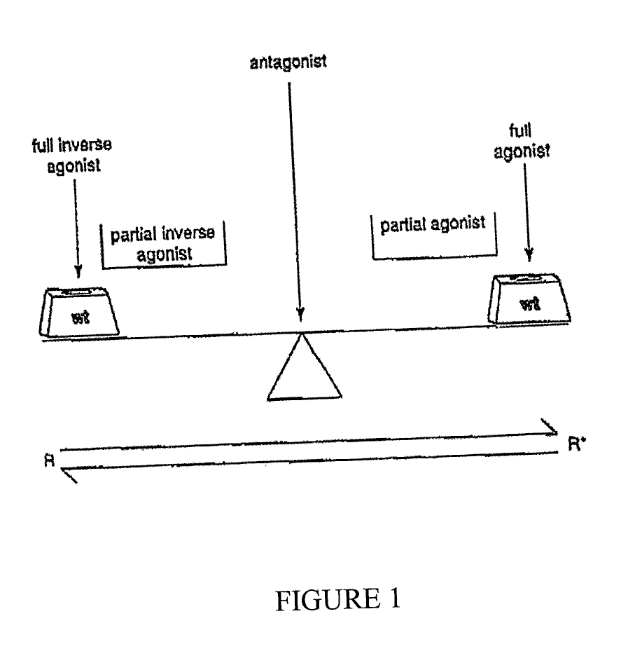

In a specific embodiment, the paradoxical pharmacologic agent is selected from the group consisting of an agonist, an antagonist and an inverse agonist. A receptor is a component of a cell that interacts selectively with an extracellular compound to initiate a biochemical change or cascade of biochemical actions. Thus, binding of a drug to a receptor determines the relationship between a given dose of a drug and the effect and the selectivity of the given drug's activity and effect. A skilled artisan is cognizant that drugs acting at G protein-coupled receptors display a spectrum of efficacies. In the traditional receptor theory, it is assumed that the unliganded receptor is quiescent. Binding of an agonist induces a conformational change with causes the receptor to activate the G-protein. However, in the more recent two-state model, it is understood that the position of the equilibrium between the inactive and the active state of the receptor varies with individual receptors and is altered by the presence of receptor ligands. For example, agonists stabilize the active state, while inverse agonists stabilize the inactive state. The theory of the equilibrium between states is best described as a see-saw in FIG. 1. A continuum of ligands between fall agonists (the extreme right-hand side) and full inverse agonists (the extreme left-hand side) with antagonists defining the position of the fulcrum.

A specific embodiment of the present invention comprises administering the paradoxical pharmacologic agent to an animal. Specifically, the animal is a human. Other animals that may be treated using the method of the invention include, but are not limited to monkeys, cows, horses, pigs, dogs, cats, sheep goats, rabbits, rats, mice, birds, chickens or fish.

In another specific embodiment of the present invention, the paradoxical pharmacologic agent is administered to a mucosal surface of said animal. More specifically, the mucosal surfaces include, but are not limited to, intranasal surface, oral surface, gastrointestinal surface, and genitourinary tract surface.

In a further specific embodiment, the paradoxical phannacologic agent is administered parenterally to the animal. In particular, the parenteral administration is intraperitoneal, intravenous, subcutaneous, intramuscular and intradermal.

One skilled in the art recognizes that the route of administration is an important determinant of the rate of efficiency of absorption. For example, the alimentary route, e.g., oral, rectal, sublingual and buccal, is generally considered the safest route of administration. The delivery of the drugs into the circulation is slow, thus eliminating rapid, high blood levels of the drug that could potentially have adverse effects. Although this is considered the safest route of administration, there are several disadvantages. One important disadvantage is that the rate of absorption varies, which is a significant problem if a small range in blood levels separates a drug's desired therapeutic effect from its toxic effect. Also, patient compliance is not always ensured. Furthermore, with oral administration, extensive hepatic metabolism may occur before the drug reaches its target site. Another route of administration is parenteral, which bypasses the alimentary tract. One important advantage of parenteral administration is that the time for the drug to reach its target site is decreased resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions, however, there is usually little systemic absorption. Inhalation of a drug provides rapid access to the circulation and is the common route of administration for gaseous and volatile drugs. It is also a desired route of administration when the targets are present in the pulmonary system.

In a specific embodiment, the paradoxical pharmacologic agent is a prophylactic treatment. More specifically, the prophylactic treatment requires chronic administration of the paradoxical pharmacologic agent. A skilled artisan is cognizant that the length of time involved in chronic administration can vary with the drug that is being utilized and the desired effect. Furthermore, one skilled in the art realizes that an acute agonist exposure may produce activation of receptors and increased signaling, while chronic exposure may produce desensitization and decreased signaling.

A specific embodiment of the present invention is that the paradoxical pharmacologic agent is given in a daily, dose, or multiple times a day, depending on its half-life. In further specific embodiments, the paradoxical pharmacologic agent is given in a single dose. The single dose may be administered daily, or multiple times a day, depending on its half-life, weekly, or multiple times a week, or monthly or multiple times a month. In a further embodiment, the paradoxical pharmacologic agent is given in a series of doses. The series of doses may be administered daily, or multiple times a day, depending upon its half-life, weekly, or multiple times a week, or monthly, or multiple times a month.

One skilled in the art of pharmacokinetics will recognize the importance of understanding the bioavailability and the half-life of the drug in relation to dosing of the particular drug. It is well known that a drug accumulates in the body if the time interval between doses is less than four of its half-lives, in which case, the total body stores of the drug are increased exponentially to a plateau or steady-state concentration. The average total body store of a drug at the plateau is a function of the dose, the interval of the doses, the bioavailability and the elimination of the drug. Thus, a skilled artisan is capable of determining the dose and interval of the dose for a given drug to achieve the desired effect.

Another specific embodiment of the present invention is a method of treatment of hypertension comprising the step of administering to an animal a paradoxical pharmacologic agent, wherein said agent restores compensatory mechanisms or activates redundant mechanisms. In specific embodiments, the paradoxical pharmacologic agent is an agonist or an inverse agonist. More particularly, the agonist is a pressor agent. Exemplary pressor agents include, but are not limited to, phenylephrine, angiotensin II, epinephrine or norepinephrine.

Furthermore, it is well within the scope of the present invention to include modified agonists, modified antagonists or modified inverse agonists. Modified agonists, modified antagonists or modified inverse agonists include, but are not limited to, genetic or chemical modifications to known and unknown agonists, antagonists or inverse agonists which increase or decrease their efficacy. These type of modifications are well known and practiced in the art, e.g., genetic mutagenesis or chemical mutagenesis.

Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

In to using mutagenesis techniques to produce modified agonists, modified antagonists or modified inverse agonists as discussed above, the present inventor also contemplates that structurally similar compounds may be formulated to mimic the key portions of the agonists, antagonists or inverse agonists. Such compounds, which may be termed peptidomimetics, may be used in the same manner as the agonists, antagonists or inverse agonists of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998)). A structural motif that appears to be evolutionarily conserved in certain toxins is small (30-40 amino acids), stable, and high permissive for mutation. This motif is composed of a beta sheet and an alpha helix bridged in the interior core by three disulfides.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

In a specific embodiment, the pressor agent is a prophylactic treatment. More specifically, the prophylactic treatment requires chronic administration of the pressor agent. Because pressor agents cause initial increases in blood pressure, they are contraindicated in hypertensive patients. With chronic treatment, these pressor agents may prevent hypertension and reverse the elevated blood pressure in established hypertension. One skilled in the art recognizes that this pharmacologic treatment mimics the body's response to exercise. Thus, it is within the scope of the present invention that chronic administration includes chronic-intermittent administration and any variations of the like.

### Dosage and Formulation

Aqueous compositions of the drugs (agonist, antagonist and inverse agonists) that are in present invention comprise an effective amount of the chemical compound or pharmaceutically acceptable salts thereof dissolved and/or dispersed in a pharmaceutically acceptable carrier and/or aqueous medium. One skilled in the art is cognizant that the phrases “pharmaceutically and/or pharmacologically acceptable” refer to molecular entities and/or compositions that do not produce an adverse, allergic and/or other untoward reaction when administered to an animal, as appropriate. Exemplary pharmaceutically acceptable carriers include, but are not limited to, any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For administration of any of the compounds used in the present invention, preparations should meet sterility, pyrogenicity, general safety and/or purity standards as required by FDA Office of Biologics standards.

The compounds may generally be formulated for parenteral administration, e.g., formulated for injection via the intravenous, intramuscular, subcutaneous, intralesional, and/or even intraperitoneal routes. The preparation of an aqueous compositions that contain an effective amount of the chemical compound or pharmaceutically acceptable salts thereof as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessanies. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

These methods described herein are by no means all-inclusive, and further methods to suit the specific application will be apparent to the ordinary skilled artisan. Moreover, the effective amount of the compositions can be further approximated through analogy to compounds known to exert the desired effect.

The following examples are offered by way of example, and are not intended to limit the scope of the invention in any manner.

### EXAMPLE 1

### The Use of Pressor Agents in Hypertension

The spontaneously hypertensive rat (SHR) is a widely used experimental model of hypertension. This strain is genetically pre-disposed to develop high blood pressure. At the age of six weeks, these rats are not hypertensive; but they develop elevation in blood pressure with age and they become fully hypertensive at the age of 12 weeks.

Male, spontaneously hypertensive rats (SHR) were acclimated for at least 2 weeks before obtaining 24 hour baseline measurements of blood pressure. Baseline estimates of systolic arterial pressure were then obtained using the tail-cuff method.

Animals were treated with equal ratios of norepinephrine (NE) and epinephrine (E) (50 ng/min/rat of E and 50 ng/min/rat of NE) beginning at 6 weeks of age. The drugs were infused in a chronic manner via an osmotic mini-pump (ALZA) for 2 hours/day, 5 days a week for 28 days. Control animals were infused in a similar manner, however, the solution that was infused only contained a vehicle, no drugs. After infusion of the pressor drugs for 28 days, blood pressure was measured via the tail-cuff method on day 28, 2 days after and 3 days after infusion. The 2 day post-pump measurements were made to ensure that no drug was being delivered at the time of the measurement.

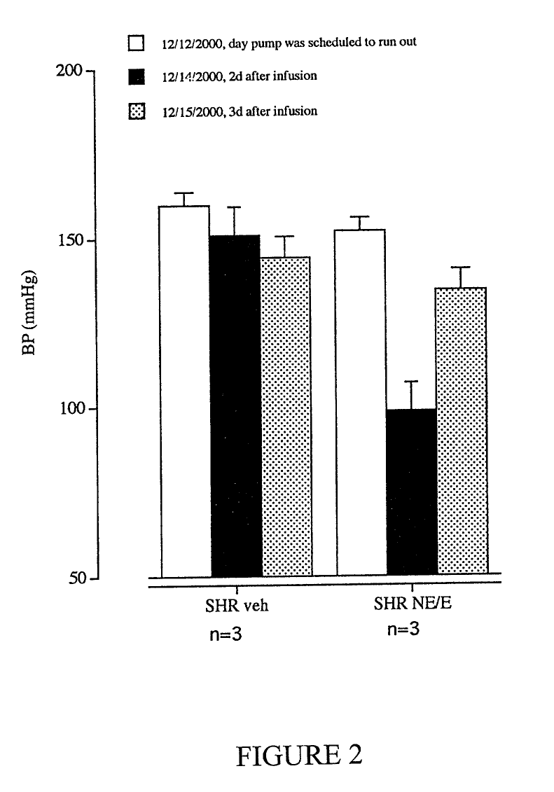

FIG. 2 shows that 2 days after the 28 days of the infusion of NE/E, the mean blood pressure was 100 mmHg in the NE/E group compared to 150 mmHg in the control group. This decrease in blood pressure was short lived because by day 3 after the infusion the blood pressure in the NE/E group was similar to the blood pressure in the control group.

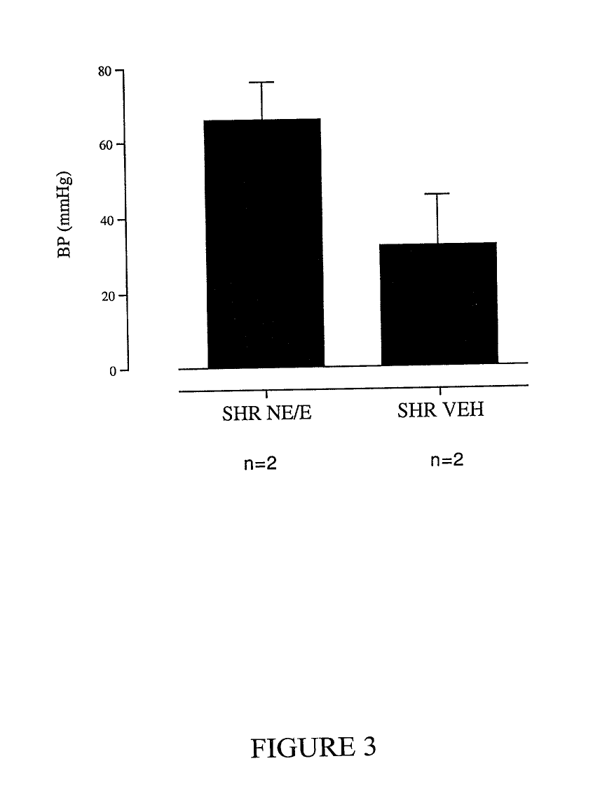

To determine a possible mechanism underlying the decrease in blood pressure after infusion of NE/E, L-NAME (N(omega)-nitro-L-arginine methyl ester), which is a nitric oxide synthase inhibitor, was infused at a rate of 10 ml/kg/10 min for a total 10 min. After infusion of the L-NAME, blood pressure was measured via the tail-cuff method. FIG. 3 illustrates the effect of the blood pressure after infusion of L-NAME.

Nitric oxide is tonically released by the activity of the constitutive endothelial and neuronal NO synthases (NOS). Nitric oxide is vasodilatory, and tonically produced NO maintains low blood pressure. During exercise, there is an increase in norepinephrine and an increase in nitric oxide production. Thus, these results suggest that the increase in norepinephrine and epinephrine also result in a concomitant increase in the production of nitric oxide. The increase in production of nitric oxide is due to an activation of nitric oxide synthase, which in turn increase the levels of nitric oxide. The addition of L-NAME inhibits the nitric oxide synthase resulting in an increase in blood pressure in the NE/E treated animals due to the fact that these animals were using the NO as rapidly as it was being synthesized. In the controls, the further decrease in the blood pressure is likely due the endogenous levels of NO.

Thus, one skilled in the art will recognize that this treatment restores the elevated blood pressure of SHR rats. Yet further, one skilled in the art will recognize that this type of intermittent infusion of pressor agents mimics exercise resulting in a net decrease in blood pressure in the SHR rats.

One skilled in the realizes that these results provide an example of using a paradoxical pharmacologic agent to elicit a compensatory mechanism.

### EXAMPLE 2

### The Use Insulin Antagonists or Inverse Agonists in the Treatment of Diabetes

The streptoxocin-induced rat model of diabetes and an obese Zucker rat model are used. Male streptoxocin-induced rats, the Zucker rats and control male rata are acclimated for at least 1 day before obtaining 24 hour baseline measurements of insulin and glucose blood levels. An insulin receptor antagonist is infused in a chronic or chronicintermittent manner into control rats and the streptoxocin-induced and Zucker rats via an osmotic mini-pump (ALZA) for 28 days. Several doses are used to incrementally increase the plasma concentration of the antagonist. After infusion of antagonist for 28 days, blood levels of glucose and insulin are measured.

Chronic infusions of an insulin receptor antagonist and/or an insulin receptor agonist may lower the elevated blood glucose levels in the rat models of diabetes.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Arnold S E, Franz B R, Trojanowski J Q, Elderly patients with 78 schizophrenia exhibit infrequent neurodegenerative lesions. Neurobiol of Aging 1994; 15: 299-303.

Arnold S E, Trojanowski J Q, Gur R E, Blackwell P, Han L-Y, Choi C, Absence of neurodegeneration and neural injury in the cerebral cortex in a sample of elderly patients with schizophrenia. Arch Gen Psych 1998; 55: 225-32.

Bristow M R, Ginsburg R, Minobe W, Cubicciotti R S, Sageman W S, Lurie K, Billingham M E, Harrison D C, Stinson E B, Decreased catecholamine sensitivity and β-adrenergic receptor density in failing human hearts. N Engl J Med 1982;307:205-11.

Hjalmarson A, Goldstein S, Fagerberg B, Wedel H, Waagstein F, Kjekshus J, Wikstrand J, El Allaf D, Vitovec J, Aldershvile J, Halinen M, Dietz R, Neuhaus K L, Janosi A, Thorgeirsson G, Dunselman P H, Gullestad L, Kuch J, Herlitz J, Rickenbacher P, Ball S, Gottlieb S, Deedwania PEffects of controlled-release metoprolol on total mortality, hospitalizations, and well-being in patients with heart failure: the Metoprolol CR/XL Randomized Intervention Trial in congestive heart failure (MERIT-HF). MERIT-HF Study Group. JAMA 2000; 283: 1295-302.

Joyce J N, Myers A J, Gurevich E, Dopamine D2 bands in normal human temporal cortex are absent in Alzheimer's disease. Brain Res 1998; 784: 7-17.

Packer M, Bristow M R, Cohn J N, Colucci W S, Fowler M B, Gilbert E M, Shusterman N H, The effect of carvedilol on morbidity and mortality in patients with chronic heart failure. N Engl J Med 1996; 334: 1351-5.

Walker Z, Costa D C, Ince P, McKeith I G, Katona C L, In-vivo demonstration of dopaminergic degeneration in dementia with Lewy bodies. Lancet 1999; 354: 646-7.

Zubenko G S, Hughes H B, Stiffer J S, Neurobiological correlates of a putative risk allele for Alzheimer's disease on chromosome 12q. Neurology 1999; 52: 725-32.

One of skill in the art readily appreciates that the present invention is well adapted to carry out the objectives and obtain the ends and advantages mentioned as well as those inherent therein. Compositions, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. A method of treatment of a disease comprising the step of administering to an animal a paradoxical pharmacologic agent, wherein said agent restores compensatory mechanisms or activates redundant mechanisms.

2. The method of claim 1, wherein, said disease is selected from the group consisting of hypertension and diabetes.

3. The method of claim 1, wherein said paradoxical pharmacologic agent is selected from the group consisting of an agonist, an antagonist or an inverse agonist.

4. The method of claim 1, wherein said paradoxical pharmacologic agent is administered to a mucosal surface of said animal.

5. The method of claim 1, wherein said paradoxical pharmacologic agent is administered parenterally to said animal.

6. The method of claim 1, wherein said paradoxical pharmacologic agent is a prophylactic treatment.

7. The method of claim 6, wherein said prophylactic treatment requires chronic administration of said paradoxical pharmacologic agent.

8. The method of claim 1, wherein said paradoxical pharmacologic agent is given in a single dose.

9. The method of claim 1, wherein said paradoxical pharmacologic agent is given in a daily dose.

10. The method of claim 1, wherein said paradoxical pharmacologic agent is given in a series of doses.

11. The method of claims 1, wherein said method further comprises the administration of a combination of at least one paradoxical pharmacologic agent and at least one non-paradoxical pharmacologic agent.

12. A method of treatment of hypertension comprising the step of administering to an animal a paradoxical pharmacologic agent, wherein said agent restores compensatory mechanisms or activates redundant mechanisms.

13. The method of claim 12, wherein said paradoxical pharmacologic agent is an agonist or an inverse agonist.

14. The method of claim 13, wherein said agonist is a pressor agent.

15. The method of claim 14, wherein said pressor agent is selected from the group consisting of phenylephrine, angiotensin II, epinephrine and norepinephrine.

16. The method of claim 14, wherein said pressor agent is a prophylactic treatment of hypertension.

17. The method of claim 16, wherein said prophylactic treatment requires chronic administration of said paradoxical pharmacologic agent.

18. The method of claim 12, wherein said method further comprises the administration of a combination of at least one paradoxical pharmacologic agent and at least one non-paradoxical pharmacologic agent.
